(19) United States
(12) Patent Application Publication
(10) Pub. No.: US 2004/0072221 A1
(43) Pub. Date: Apr. 15, 2004

(54) Process for the isolation of the extrachromosomal dna and method for the diagnosis based on the electrophoretic analysis of such dna

(76) Inventor: Maria Giulia Menesini, Siena (IT)

Correspondence Address: Nixon & Vanderhye, 8th Floor, 1100 North Glebe Road, Arlington, VA, 22201-4714, US

(21) Appl. No.: 10/467,617

(22) PCT Filed: Feb. 7, 2002
(86) PCT No.: PCT/IT02/00075

(30) Foreign Application Priority Data
Feb. 8, 2001 (IT) SI2001A000002

(51) Int. Cl. C12Q 1/68; C12N 1/08

(52) U.S. Cl. 435/6; 435/270

(57) ABSTRACT

A process is disclosed for the extraction and isolation of the extrachromosomal DNA from blood and cells with a view to detecting DNA alterations linked to a possible pathology in progress and making a diagnosis. The process for DNA extraction comprises the use of a buffer, a heat treatment, followed by treatment with a detergent and a salt. The extract thus obtained is suitable for undergoing electrphoresis on agarose gel.

The present invention relates to a process for the extraction and isolation of extrachromosomal DNA at an adequate degree of purity, from a suitable biological sample withdrawn from a mammal and in particular from a human being. The invention also relates to an electrophoretic diagnostic method for detecting possible alterations of pathological consequence in the extrachromosomal DNA extracted and isolated by means of the process of the present invention, with a view to confirming or denying the suspect that the above mentioned mammal, or, in particular, the human being might be affected by a pathological form, whose presence could be diagnosed observing an alteration, inborn or induced, of its DNA, analysis that, in short, is known with the English expression “DNA diagnosis”.

For an in depth report on DNA diagnosis, see, for example, chapter 40, Medical Genetics, in “Current Medical Diagnosis and Treatment”, 38^{th} edition (1999), edited by Lawrence M. Tierney, Stephen J. Mc Phee and Maxine A, Papadakis and published by Appleton and Lange, publication which is incorporated by reference in the present description.

According to the present invention, by “suitable biologic sample” is meant a sample consisting of (a) whole blood or plasma/serum, or (b) the buffy coat fraction obtained by centrifuging heparinized blood or (c) the homogenate of tissues and cells. (It is known that by “buffy coat” is meant the layer consisting of leukocytes and platelets which forms between the layer of red blood cells and plasma by centrifuging whole blood).

It has long since been known that nucleic acids, in particular deoxyribonucleic acid (or DNA), constitute the key elements for the transmission of genetic information in all living organisms. DNA is comprised of a sequence of nitrogen bases: Adenine (A), Cytosine (C), Guanine (G) and Thymine (T). The sequences of nitrogen bases varying from a few units to millions of units in length, form the so-called genes whose functions have been identified and described in detail in the scientific literature. The research on genoma, the pool of all genes that characterize any specific species, aims at identifying the normal sequence of genes. Many genes have been isolated and identified, and various diseases have been characterized, brought about by their abnormalities. The research on the genoma, however, is still at a beginning stage because there are still hundred thousands of genes waiting for isolation and identification.

Circulating DNA study has recently prompted an increasing interest (see Anker et al, Cancer Metastasis Rev. 1999, 18:65-73). To know this DNA means to obtain viable means enabling early diagnosis of various diseases, for example, the onset of cancer, by non-invasive method.

Some genetic alterations have been identified and correlated with defined diseases using analytical methods such as Polymerase Chain Reaction (PCR), the Fluorescence in situ Hybridization (FISH), the in situ PCR (isPCR), the quantitative PCR, the sequence of circulating DNA and the RAS mutation and so on. For all of these analyses, it is necessary to have available a sample of isolated and purified circulating DNA/RNA, suitable for the object. It is well known that from a sample of blood plasma, serum, erythrocytes, leukocytes etc. can be obtained. The difficulty presented during the extraction of DNA from these fractions was mainly attributed to their high content of proteins and the possible contamination by hemoglobin which is considered harmful for the outcome of PCR analysis. To date various extraction methods are known but the DNA/RNA thus obtained often presents undefined or even unknown properties. Furthermore, these types of analyses, not only entail high cost and prolonged times, but are complicated and very often furnish uncertain results. The incidence is around 30-60% only.

Therefore, it is necessary to provide a simple, reliable and rapid method to detect the DNA/RNA pattern in blood samples useful for the identification of DNA alterations and for the diagnosis of various related pathologies.

The essence of the present invention is based on the casual observation of a blood sample obtained from a calf affected by muscle dystrophy.

The extraction of circulating DNA with various methods shows that in a determined process DNA patterns with dear bands on agarose gel are obtained. It was observed that the DNA pattern of an autoimmune subject is different from that of an healthy individual. We postulated that the above mentioned observations should have more than a diagnostic value.

The main object of the present invention is to provide a process of extraction and isolation of extrachromosomal DNA instead of chromosomal DNA from a biological sample withdrawn from a mammal, in particular from a human being. The process should be rapid, simple and unexpensive and should also provide a warranty of obtaining isolated DNA of suitable purity, specially guaranteeing the absence of relevant amounts of contaminating substances such as the residual proteins and hemoglobin which could alter the outcome of electrophoretic analysis whereat the isolated DNA is submitted for diagnostic purposes.

A further object of the present invention is to provide a simple and reliable electrophoretic diagnostic method for detecting the eventual alterations of pathological significance in the DNA extracted and isolated, according to the above mentioned process.

According to the invention, the process of extraction and isolation of the extrachromosomal DNA comprises subjecting a biological sample withdrawn from a mammal and consisting of (a) whole blood or plasma/serum, or (b) a “buffy coat” fraction obtained by centrifuging heparinized blood, or (c) homogenized tissues or cells, to the following steps:

(1) adding a buffer to said biological sample;

(2) heating the resulting mixture to a temperature of about 25-100° C.;

(3) centrifuging the product of step (2) and removing the supernatant;

(4) adding a detergent to the supernatant and maintaining the resulting mixture at about 25° C. for 4-8 minutes;

(5) adding to the mixture of step (4) a salt selected from the halides of alkali metals or alkaline-earth metals and keeping the resulting mixture under stirring for about 8-12 minutes;

(6) centrifuging the mixture of step (5) and removing the extrachromosomal DNA-containing supernatant; and, optionally,

(7) concentrating said supernatant via known methods.

For the purposes of the invention it is obviously particularly relevant the case in which the above mentioned mammal is a human being, whose thus isolated extrachromosomal DNA is suitable for undergoing “DNA diagnosis”.

There are various reagents which could be appropriately utilized as buffers [in step (1)] and detergents [in step (4)]. These buffers and detergents are well known to the experts in the techniques and methods used in the molecular biology laboratories.

A non-limiting example of buffer is the 2-amino-2-aiydroxymethyl)-1,3-propandiol, abbreviated as TBS, or tris buffer saline (pH=7.8).

A non-limiting example of detergent is sodium dodecylsulfate (SDS).

The salt in the step (5) is appropriately selected from the alkali metal chlorides and it is, preferably, potassium chloride.

The electrophoretic diagnostic method according to the invention for detecting possible alterations of pathological importance in the extrachromosomal DNA of a mammal suspected of being affected by a pathology hereinbelow the “first mammal”) comprises the steps consisting of:

(i) extracting and isolating by means of the above mentioned process of extraction and isolation from a biological sample withdrawn from the first mammal a quantity of extrachromosomal DNA sufficient for electrophoretically analyzing such DNA on agarose gel via known methods, thus obtaining a first electrophoretic map;

(ii) extracting and isolating by means of the above mentioned process of extraction and isolation from a biological sample withdrawn from a healthy mammal of the same species of the first mammal (hereinbelow the “second mammal”) a quantity of extrachromosomal DNA sufficient for electrophoretically analyzing such DNA on agarose gel via known methods, thus obtaining a second electrophoretic map; and

(iii) comparing the first with the second electrophoretic map for detecting possible alterations of the extrachromosomal DNA of the first mammal with respect to that of the second mammal and evaluating the importance and the possible pathologic nature of such alterations.

To the ultimate aims of the invention, it is obviously particularly important the case wherein the first and the second mammal are human beings. Moreover, it is particularly useful that such human beings are in a parental relationship to each other.

The method is particularly even though non exclusively useful, for the accurate diagnosis of pathologies, possibly including also the onset stages of serious diseases, such as autoimmune or tumor diseases.

The present invention shall be now described in more details in the following examples for illustrative, non limiting purposes. Furthermore, variations and modifications can be made by the experts without thereby departing from the spirit and scope of the invention.

### EXAMPLE 1

### Isolation of DNA from Whole Blood or Plasma/Serum

To 0.2-2 ml of whole blood or plasma/serum 0.1-2 volumes of buffer (preferably TBS, Tris buffer saline pH 7.8) were added. After heating to 25-100° C. (for example, by boiling for a few minutes), the sample was centrifuged at 13,000 rpm at 25° C. for 5 minutes. The supernatant thus obtained was then treated with a detergent (preferably SDS; sodium dodecylsulfate, in TBS, at the final concentration of 2%) for 5 minutes at 25° C. To this mixture KCl (from 0.1 to 6 M) was added and the resulting mixture, after agitation, was kept at 25° C. for other 10 minutes. Following centrifugation at 13,000 rpm at room temperature for 10-20 minutes the supernatant thus obtained was ready for undergoing electrophoretic analysis on agarose gel (0.4-2%). It is worthwhile noting that, if necessary, the sample could be subjected to further purification by means of the currently known methods, or the end product could be concentrated via well known procedures.

The end product is generally a liquid. The electrophoretic map, in the case of a sample of a healthy human being, analyzed on the agarose gel (0.4%), shows bands having apparent molecular weight from 2 to 23 Kbs for plasma and serum and from 0.5 to 33 Kbs for whole blood.

### EXAMPLE 2

### Isolation of DNA from Nucleated Blood Cells or Homogenized Tissue

In order to isolate DNA from nucleated blood cells, heparinized blood (5-10 ml) was centrifuged at 3,000 rpm at room temperature for 3 minutes. The white layer, between plasma and red cells, hereinbelow the “Buffy Coat” (BC), was isolated with care, avoiding any possible contamination with either plasma or red cells. The BC thus obtained was suitable for undergoing the treatment described in Example 1.

For tissues or tumor cells in vitro a quantity of material was homogenized in an equivalent volume of buffer (preferably TBS) for about 5-10 minutes and was thus suitable for undergoing the treatment described in Example 1:

Under normal conditions the end product isolated from nucleated blood cells, is a clear and odorless liquid. The DNA pattern analyzed (for example from a human sample) by electrophoresis on 0.4% agarose gel shows bands from 0.5 to 33 Kbs for the nucleated blood cells; for other types of cells the DNA pattern varies according to the sample under study.

As just described above, the basic concept of the present invention consists in identifying and isolating extrachromosomal DNA. The rate at which it has been possible to isolate the DNA present in the samples under study supports the validity and reliability of the process.

Using the process of the invention it was possible, for example, to observe that the DNA pattern extracted from the BC of a healthy calf is very different from that extracted from the BC of a calf affected by muscular dystrophy. Similar results were also obtained from human samples, for example in the case of a patient who suffered from an autoimmune disease. Relevant data show that the process of present invention allows the DNA pattern alterations linked to a determined disease be detected through the modifications of its electrophoretic properties and also of its chimico-physical ones. This clearly indicates that the present invention provides a useful tool to clinical researchers for the mutated genes identification and for the diagnosis of the diseases caused by such mutations.

1. A process for the extraction and isolation of extrachromosomal DNA from a biological sample withdrawn from a mammal consisting of (a) whole blood or plasma/serum, or (b) the buffy coat fraction obtained by centrifuging heparinized blood or (c) the homogenate of tissues and cells, which comprises the steps of:
(1) adding a buffer to said biological sample;
(2) heating the resulting mixture to a temperature of about 25-100° C.;
(3) centrifuging the product of step (2) and removing the supernatant;
(4) adding a detergent to the supernatant and maintaining the resulting mixture at about 25° C. for 4-8 minutes;
(5) adding to the mixture of step (4) a salt selected from the halides of alkali metals or alkaline-earth metals and keeping the resulting mixture under stirring for about 8-12 minutes;
(6) centrifuging the mixture of step (5) and removing the extrachromosomal DNA-containing supernatant; and, optionally,
(7) concentrating said supernatant via known methods.

2. The process of claim 1, wherein the biological sample is withdrawn from a human being.

3. The process of claim 1, wherein the buffer is TBS, tris buffer saline (pH=7.8).

4. The process of claim 1, wherein the detergent is sodium dodecylsulfate.

5. The process of claim 1, wherein the salt of step (5) is selected from the alkali-metal chlorides and is preferably potassium chloride.

6. An electrophoretic diagnostic method for detecting possible alterations of pathological importance in the extrachromosomal DNA of a mammal suspected of being affected by a pathology (hereinbelow the “first mammal”) which comprises the steps of:
(i) extracting and isolating by means of the process of claim 1 from a biological sample withdrawn from said first mammal a quantity of extrachromosomal DNA sufficient for electrophoretically analyzing such DNA on agarose gel via known methods, obtaining a first electrophoretic map;
(ii) extracting and isolating by means of the process of claim 1 from a biological sample withdrawn from a healthy mammal of the same species of the first mammal (hereinbelow the “second mammal”) a quantity of extrachromosomal DNA sufficient for electrophoretically analyzing such DNA on agarose gel via known methods, obtaining a second electrophoretic map; and
(iii) comparing the first with the second electrophoretic map for detecting possible alterations of the extrachromosomal DNA of the first mammal with respect to that of the second mammal and evaluating the importance and the possible pathologic nature of such alterations.

7. The method of claim 6, wherein said first and second mammal are human beings.

8. The method of claim 7, wherein said human beings are in parental relationship to each other.

9. The method of claim 6, wherein the pathology is an autoimmune or tumor disease.

10. A sample of extrachromosomal DNA as obtained via the process of claims 1-5.

11. A sample of extrachromosomal DNA isolated from plasma of a healthy human being showing bands from 2 to 23 Kbs on 0.4% agarose gel.

12. A sample of extrachromosomal DNA isolated from whole blood or nucleated blood cells (BC) of a healthy human being showing bands from 0.5 to 33 Kbs on 0.4% agarose gel.

13. Use of a sample of claims 10-12, as a reference standard in the method of claims 6 to 9.
